(19) United States
(12) Patent Application Publication
(10) Pub. No.: US 2026/0125724 A1
(43) Pub. Date: May 7, 2026

(54) METHOD FOR MANUFACTURING FERMENTATION PRODUCTS WITH A SENSOR DEVICE

(71) Applicant: AJINOMOTO CO., INC., Tokyo (JP)

(72) Inventors: Motohiro TAKENAKA, Kawasaki-shi (JP); Masaaki FUJIE, Kawasaki-shi (JP); Yohei KODAMA, Kawasaki-shi (JP)

(73) Assignee: AJINOMOTO CO., INC., Tokyo (JP)

(21) Appl. No.: 19/424,930

(22) Filed: Dec. 18, 2025

Related U.S. Application Data
(62) Division of application No. 17/807,998, filed on Jun. 21, 2022.
Application No. 17/807,998 is a continuation of application No. PCT/JP2020/044859, filed on Dec. 2, 2020.

(30) Foreign Application Priority Data
Dec. 25, 2019 (JP) 2019-233846

(51) Int. Cl. C12P 13/12; C12M 1/00; C12M 1/04; C12M 1/34; G01N 21/3577; G01N 21/359

(52) U.S. Cl. CPC C12P 13/12; C12M 27/04; C12M 29/04; C12M 29/08; C12M 41/30; G01N 21/3577; G01N 21/359

(57) ABSTRACT

The present invention is a manufacturing method for producing fermentation products using a fermentation vessel, including steps of: preparing a fermentation vessel and a sensor, introducing liquid into the fermentation vessel, and operating the fermentation vessel in which properties of liquid in the fermentation vessel are measured to adjust operating conditions; wherein the sensor device has a sensor for measuring liquid properties and a sensor cover body; a bottom permeable portion for passing liquid and crystals in the liquid is disposed on the bottom surface of the cover body, and a top permeable portion for passing liquid and crystals in the liquid is disposed on the top surface of the cover body; micropores are respectively formed in the bottom permeable portion and the top permeable portion; and micropores disposed in the top permeable portion are the same or larger than micropores disposed in the top permeable portion.

### CROSS-REFERENCE TO RELATED APPLICATIONS

The present application is a divisional of U.S. application Ser. No. 17/807,998, filed on Jun. 21, 2022, which is a continuation of International Patent Application PCT/JP2020/044859, filed Dec. 2, 2020, which is based on and claims the benefit of priority to Japanese Application No. 2019-233846, filed Dec. 25, 2019. The entire contents of all the above applications are incorporated herein by reference.

### TECHNICAL FIELD

The present invention pertains to a method for producing fermentation products, and in particular to a fermentation product manufacturing method and sensor device for producing fermentation products by the fermenting operation of a fermentation vessel into which bubbles are mixed, and in which crystals of an average particle size of 5 μm or larger are produced in a liquid.

### BACKGROUND ART

The production of various fermentation products requires measurement of various properties such as the electrical conductivity and turbidity of liquid in a fermentation vessel, and the concentration of specific components in the liquid. Japanese Patent No. 3074781 (Patent Document 1) describes a method for manufacturing L-lysine. In this production method, a carbon source is added to maintain a carbon source concentration at 5 g/L or less in the culture liquid. I.e., in this method, carbon source concentration is measured by sampling the culture liquid in a timely manner and directly analyzing carbon source concentration, or by measuring pH and dissolved oxygen concentration to sense a carbon source deficiency from changes therein, so as to control feeding of the medium.

Although it is relatively easy to extract a portion of liquid to be measured from the manufacturing process and measure properties of the liquid, as in the invention described in Patent Document 1, inline measurement without extracting liquid from the manufacturing process is preferable from a manufacturing efficiency standpoint. However, it can be difficult to measure liquid properties inline, particularly if bubbles are present in the liquid to be measured, or crystals are formed in the liquid. For example, bubbles of supplied oxygen or air, or bubbles of the carbon dioxide gas metabolic product of microorganisms themselves in culture may be mixed into the culture liquid during aerated culture, introducing noise into the readings, or increasing measurement errors.

Japanese Patent No. 4420168 (Patent Document 2) describes a turbidity sensor. The turbidity sensor has a hollow semicylindrical member made of stainless steel, with a test solution inlet and an automatically opening and closing swing valve at the bottom, and a hole at the top for venting bubbles. In addition, a wetted photometric portion of a laser turbidimeter is disposed at the tip position on the inside of the hollow semicylinder. When measuring with this turbidity sensor, a swing valve is first opened to replace the test solution inside the hollow semicylinder. The swing valve is then closed, bubbles in the hollow semicylinder are discharged through bubble vent holes and, after the detected turbidity is allowed to stabilize, turbidity is measured. In the Patent Document 2 invention, the effect of bubbles in a liquid being tested is thus reduced.

### PRIOR ART DOCUMENTS

### Patent Documents

[Patent Document 1] Japanese Patent No. 3,074,781

[Patent Document 2] Japanese Patent No. 4,420,168

### SUMMARY OF INVENTION

### Problem to Be Solved

However, the turbidity sensor of Patent Document 2 requires waiting for the detection value to stabilize after the swing valve is closed, making it difficult to perform real-time detection. If the turbidity sensor is applied to a liquid in which crystals form in the liquid, there is a risk that crystals may accumulate on moving parts such as the swing valve, causing failures. In addition, the requirement in the Patent Document 2 turbidity sensor for a swing valve which is opened and closed by remote control complicates the structure, leading to the problem of time-consuming maintenance to achieve stable operation over long durations.

On the other hand, when a sensor is applied to a liquid in which crystals are produced, there is a need not only to suppress the effect of the crystals, but also to analyze, simultaneously with the liquid phase, the amount of solids precipitated when the concentration of the fermentation product in the liquid phase exceeds its solubility. Here, when a sensor cover is provided on the sensor to suppress the effect of bubbles, crystals are deposited near the sensor and inside the sensor cover, making it difficult to accurately measure the concentration of products. Measures such as discharging crystals precipitated inside the sensor cover as needed are therefore required to keep the crystal concentration in the fermentation vessel and the crystal concentration around the sensor at the same levels, so as to prevent such deposition.

The present invention therefore has the object of providing a fermentation product manufacturing method and sensor device for same with which, using a simple structure, the effects on measurement results by bubbles mixed into a liquid are suppressed, deposition of crystals produced in the liquid around the sensor and within the sensor cover is prevented, properties of liquid and solids within a fermenting vessel are measured and fermentation operations can be conducted based on those measurements.

### Solution Means

To solve the above-described problems, the present invention is a method for manufacturing fermentation products in which the fermentation products are produced by fermentation operation of a fermentation vessel in which bubbles are mixed and crystals with an average particle size of 5 μm or greater are formed in a liquid, comprising steps of: preparing a fermentation vessel and a sensor device for measuring properties of liquid inside the fermentation vessel; introducing liquid to be fermented into the fermentation vessel; and operating the fermentation vessel for fermentation, wherein properties of liquid in the fermentation vessel are measured by the sensor device, and conditions of fermentation operation are adjusted based on results of the measurement; wherein the sensor device includes a sensor for measuring liquid properties and a cover body for the sensor disposed to surround the sensor; wherein the cover body includes a bottom side permeable portion for passing at least a portion of the liquid and crystals in the liquid disposed on at least a portion of the bottom surface of the cover body and a top side permeable portion for passing at least a portion of the liquid and crystals in the liquid disposed on at least a portion of the top surface of the cover body; and wherein the bottom side permeable portion and the top side permeable portion have numerous micropores for passing liquid respectively, and the micropores disposed on the top side permeable portion are sized to be the same as or larger than the micropores disposed on the bottom side permeable portion.

In the invention thus constituted, the sensor device for measuring properties of liquid in the fermentation vessel has a cover body disposed so as to surround the sensor. Bottom and top permeable portions through which a liquid and at least some of the crystals in the liquid can pass is provided on this cover body, therefore liquid to be measured is able to constantly flow in and out of the cover body. Liquid inside the cover body is therefore constantly replaced, and sensors disposed on the cover body can continuously measure liquid properties in real time. Because the bottom and top permeable portions of the cover body allow at least some of the crystals in the liquid to pass through, crystals formed in the liquid are less likely to accumulate in the cover body, therefore fermentation operations can continue for a long duration without performing maintenance such as cleaning the cover body. Since micropores in the top permeable portion have a size equal to or greater than micropores disposed on the bottom permeable portion, bubbles floating up from below the cover body have difficulty passing through the micropores in the bottom permeable portion, thus enabling penetration by bubbles into the cover body to be effectively suppressed. On the other hand micropores in the top permeable portion are the same or larger than those in the bottom permeable portion, therefore bubbles penetrating into the cover body can easily float up within the cover body and pass through the top permeable portion to be discharged. Bubbles penetrating into the cover body can thus be prevented from accumulating therein, so adverse effects on sensor readings can be effectively suppressed.

In the present invention the average diameter of the bubbles mixed into the fermentation vessel liquid is preferably 50 μm or greater. In the invention thus constituted, the average diameter of the bubbles mixed into the fermentation vessel liquid is 50 μm or greater, therefore the cover body can suppress penetration of bubbles while allowing passage of crystals produced in the liquid with an average particle size of 5μ or greater. Stable readings can thus be obtained by the sensor. Note that average bubble diameter in this Specification denotes the mean value of the weighted chord length (square-weighted cord length) obtained by measurement using focused beam reflectometry (FBRM), corresponding to what is known as the volumetric average value.

In the present invention the fermentation vessel preferably has a ventilation tube for feeding gas into the fermentation vessel, with a hole diameter at the outlet of the ventilation tube of 1 μm or greater.

In the invention thus constituted, the hole diameter at the outlet of the ventilation tube for feeding gases into the fermentation vessel is 1 μm or greater, thus increasing the average diameter of bubbles mixed into in the fermentation vessel liquid. The cover body can therefore suppress penetration by bubbles while allowing crystals produced in the liquid with an average particle size of 5μ or greater to pass through. Stable readings can thus be obtained by the sensor.

In the present invention the fermentation vessel preferably has a ventilation tube for feeding gas into the fermentation vessel, and the volume of gas fed into the fermentation vessel per hour through this ventilation tube is less than or equal to twice the culture medium volume at the start of fermentation in the fermentation vessel.

In the invention thus constituted, the volume of gas fed into the fermentation vessel per hour through the ventilation tube is less than or equal to twice the volume of the culture liquid at the start of fermentation in the fermentation vessel, therefore gas can be made to dissolve into the liquid without overly fragmenting the gas bubbles fed into the fermentation vessel. As a result, penetration of bubbles into the cover body can be easily suppressed.

In the present invention the cover body is preferably formed in an approximately cylindrical shape, with the sensor extending in an axial direction therein.

In the invention thus constituted, the cover body is formed in an approximately cylindrical shape, therefore bubbles floating up from the bottom of the cover body can easily flow upward along the bottom surface of the cover body, further suppressing penetration by bubbles into the cover body. Since the cover body is formed in an approximately cylindrical shape, bubbles penetrating into the cover body and floating to the surface are collected in the highest part of the cover body, where they are less likely to contact the sensor. This enables adverse effects on measurement to be minimized even if bubbles do penetrate into the cover body.

In the present invention, preferably, the bottom permeable portion is disposed on the entire surface of the lower semicircular portion of the approximately semi-cylindrical cover body, and the top permeable portion is disposed on the entire surface of the upper semicircular portion of the cover body.

In the invention thus constituted, bottom and top permeable portions are respectively disposed over the entire surfaces of the lower and upper semicircular portions, therefore the portion of the cover body through which liquid and crystals pass can be made extremely large. Crystals which have entered into the cover body can as a result be easily discharged to the outside, and accumulation of crystals inside the cover body can be suppressed.

In the present invention the cover body is preferably formed from a thin sheet of metal, and micropores disposed in the bottom and top permeable portions are approximately circular holes formed in the thin sheet of metal.

In the invention thus constituted, micropores in the bottom and top permeable portions are formed by holes disposed in a thin metal plate, therefore compared to forming the bottom and top permeable portions with a mesh made by weaving strands, crystals are less likely to adhere to and deposit on the bottom and top permeable portions, and maintainability of the sensor cover can be improved. By forming micropores of the bottom and top permeable portions using holes in a thin metal plate, the cover body can be constituted to be less susceptible to damage and more durable than a mesh object.

In the present invention the diameter of micropores in the top permeable portion is preferably one to five times the diameter of the micropores in the bottom permeable portion.

In the invention thus constituted, selection of a diameter for the micropores in the top permeable part of one to five times the diameter of the micropores of the bottom permeable part enables an appropriate balance to be struck between suppressing the penetration of bubbles into the cover body and discharging bubbles that do end up penetrating into the cover, so that the effect of bubbles on the sensor can be effectively suppressed.

In the present invention the diameter of micropores disposed in the bottom permeable portion is preferably between 540 μm and 750 μm.

In the invention thus constituted, the diameter of micropores disposed in the bottom permeable portion is between 540 μm and 750 μm, therefore penetration of bubbles large enough to easily adversely affect sensor readings can be effectively suppressed, while liquid and crystals are allowed to flow into the cover body.

In the present invention, the fermentation product produced by the fermentation operation of the fermentation vessel is preferably cysteine, and oxidation of at least part of the cysteine results in the accumulation of cystine in the fermentation vessel.

In the invention thus constituted, in the fermentation vessel, cysteine is produced as fermentation products, and cystine is formed by the oxidation of at least part of the cysteine. Therefore, in the case the diameter of micropores disposed in the bottom permeable portion is between 540 μm and 750 μm, inflow and outflow of the crystalized cysteine and cystine in the liquid through the cover body are allowed, while the entry of bubbles into cover body is effectively suppressed, the cystine concentration can be accurately measured by the sensor.

In the present invention, the cover body is preferably disposed to project diagonally downward from the sidewall surface of the fermentation vessel containing the liquid, and the measuring portion of the sensor is positioned near the tip of the cover body.

In the invention thus constituted, the cover body projects diagonally downward from the sidewall surface of the fermentation vessel containing the liquid, therefore bubbles floating up from below and reaching the cover body can easily flow upward along the bottom surface of the cover body, effectively suppressing their penetration into the cover body. Bubbles penetrating into the cover body collect at the base portion of the cover body positioned above, thus moving away from the measurement portion of the sensor positioned near the tip of the cover body, so that negative effects on measurement can be reduced.

The invention is a sensor device for measuring properties of a liquid in a fermentation vessel into which bubbles are mixed, and crystals with an average particle size of 5 μm or larger are formed in a liquid, having a sensor for measuring liquid properties, and a sensor cover body disposed to surround the sensor, whereby a bottom permeable portion for passing the liquid and at least a portion of the crystals in the liquid is disposed on at least a portion of the bottom of the cover body, and a top permeable portion for passing the liquid and at least a portion of the crystals in the liquid is disposed on at least a portion of the top of the cover body, multiple micropores for passing liquid are respectively formed on the bottom permeable portion and the top permeable portion, and the micropores disposed on the top permeable portion are the same or larger than the micropores disposed on the bottom permeable portion.

### Effect of the Invention

According to the method for manufacturing fermentation products and the sensor device used for same of the present invention, a simple structure is used to suppress the effect of bubbles mixed into the liquid on sensor readings and to prevent crystals formed in the liquid from accumulating near the sensor or in the sensor cover, so that properties of the liquid and solids in the fermentation vessel can be measured, and the fermentation operation can be performed based on those measurements.

### BRIEF DESCRIPTION OF FIGURES

FIG. 1 A cross-section showing an example of a sensor device for implementing a fermentation product manufacturing method according to the present invention, as applied to a fermentation vessel.

FIG. 2 A diagram showing the external appearance of a cover body for a sensor installed in a sensor device according to an embodiment of the present invention.

FIG. 3 A cross-section showing an expanded view of a sensor device according to an embodiment of the present invention, attached to a side wall surface of a fermentation vessel.

FIG. 4 A diagram showing an expanded view of one example of micropores formed in a cover body surface in a sensor device according to an embodiment of the present invention.

FIG. 5 A diagram schematically showing the operation of a sensor cover in a sensor device according to an embodiment of the present invention.

FIG. 6 A flow chart showing a procedure in a method for manufacturing fermentation products according to an embodiment of the invention.

FIG. 7 A diagram showing an example wherein measurement is made without use of a sensor cover.

FIG. 8 A diagram showing an example of measurement with a sensor device using an appropriate sensor cover.

FIG. 9 A diagram showing an example when measurement is performed using a sensor cover in which the micropores are too small.

FIG. 10 A diagram showing various sensor covers after use.

### MODES OF PRACTICING THE INVENTION

Below, referring to the attached figures, we explain preferred embodiments of the invention.

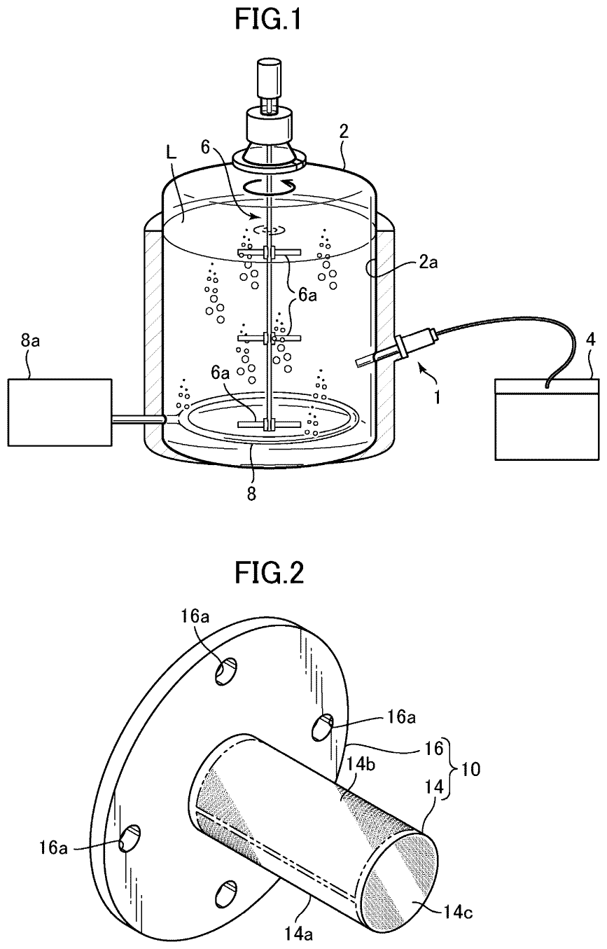

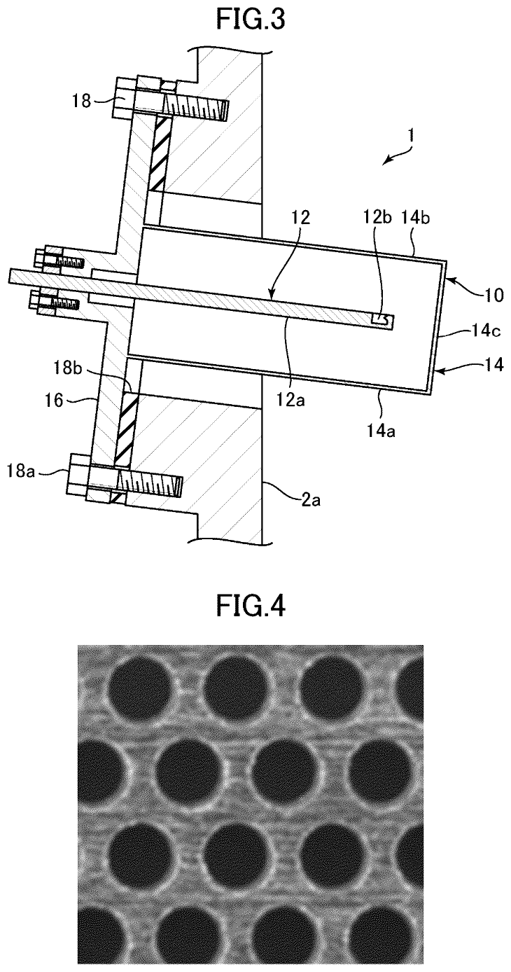

FIG. 1 is a cross-section showing an example of a sensor device for implementing a fermentation product manufacturing method according to the present invention, as applied to a fermentation vessel. FIG. 2 is a diagram showing the external appearance of a cover body for a sensor installed in a sensor device according to an embodiment of the present invention. FIG. 3 is cross-section showing an expanded view of a sensor device according to an embodiment of the present invention, attached to a side wall surface of a fermentation vessel.

As shown in FIG. 1, sensor device 1 according to an embodiment of the invention is installed on side wall 2a of fermentation vessel 2, and is constituted to make an inline measurement of amino acid concentration in a culture liquid L, which is the liquid contained in fermentation vessel 2. Furthermore, a sensor is disposed inside sensor device 1, and signals acquired by this sensor are transmitted to measurement instrument main unit 4. In the present embodiment, fermentation vessel 2 is approximately cylindrical, and is fitted with an agitator 6 along its center axis for stirring the culture liquid. Agitator 6 is furnished with multiple blades 6a for stirring culture liquid L; rotation of these blades 6a homogenizes the culture liquid L in fermentation vessel 2 by stirring.

In the present embodiment a sparger 8, being a ventilation tube, is provided on the bottom portion of fermentation vessel 2 to aerobically culture the liquid in fermentation vessel 2, and oxygen or air is introduced from supply device 8a through sparger 8 into fermentation vessel 2. Therefore fine bubbles of supplied oxygen or air or fine bubbles of carbon dioxide gas produced by the microorganisms being cultured are mixed into the culture liquid L. In the present embodiment the diameter of the hole at the outlet of gas-discharging sparger 8 is approximately 6 mm, which results in bubbles with an average diameter of 250 μm being mixed into the culture liquid in fermentation vessel 2. The hole diameter for discharging gas in the sparger 8, etc. is set at approximately 5 μm to approximately 36 μm, and bubbles with an average diameter of approximately 150 μm to approximately 350 μm are mixed into the culture liquid in fermentation vessel 2. Alternatively, a FAD (Fine Air Diffuser) can be used as an aerating tube to mix bubbles into the culture liquid. The FAD has a large number of holes ranging from approximately 1 μm to approximately 20 μm in diameter; this enables bubbles with an average diameter of approximately 50 μm to approximately 250 μm to be mixed into the culture liquid.

Referring next to FIGS. 2 and 3, we explain the constitution of a sensor device 1 according to an embodiment of the invention.

As shown in FIG. 3, sensor device 1 has a sensor cover 10 according to an embodiment of the invention, and a sensor 12 disposed inside this sensor cover.

Sensor cover 10 has an approximately cylindrical cover body 14 and a flange portion 16, disposed at the base end of this cover body 14 and affixed to the side wall surface 2a of fermentation vessel 2.

Cover body 14 is formed of a thin sheet of stainless steel with an approximately cylindrical shape, closed at the end, and is disposed to surround sensor 12. As described below, numerous micropores are disposed over the entire surface of cover body 14, and the liquid and at least some crystals in fermentation vessel 2 can pass through these micropores to flow into the cover body 14. Note that in addition to a cylinder, the cover body can also be constituted in any desired shape, such as a rectangle. The cover body can also be made of metals other than stainless steel, or resins such as polytetrafluoroethylene; it is best made of a material not easily damaged by the flow of liquid caused by stirring.

Flange portion 16 is a stainless steel disk, to the center portion of which a cover body 14 is welded to form an integrated single unit. Cover body 14 is mounted perpendicular to the flat surface of flange portion 16. Bolt holes 16a are disposed on flange portion 16, and flange portion 16 is affixed to the outside of sidewall surface 2a by affixing bolts 18a. In addition, a packing 18b is disposed between flange portion 16 and side wall surface 2a, assuring watertightness between flange portion 16 and sidewall surface 2a. Furthermore, as shown in FIG. 3, the part on the outer side of side wall surface 2a to which flange portion 16 is affixed is formed to be sloped relative to vertical. Thus when sensor device 1 is attached to the side wall surface 2a of fermentation vessel 2, cover body 14 projects diagonally downward toward the inside from the side wall surface of the container. The cover body 14 attachment angle is preferably set to suppress the inflow of bubbles into cover body 14 and to suppress pooling of liquid close to cover 14, which can lead to bacterial growth; depending on the nature of the liquid, this angle can be set close to horizontal. In the present embodiment, the center axis of cover body 14 is inclined approximately 16 degrees toward the horizontal axis.

In the present embodiment a transmission-reflectance type near-infrared spectroscopic sensor (NIR sensor) is adopted as sensor 12 to measure the concentration of amino acids in liquid L by near infrared analysis. However, in addition to NIR sensors, the sensor cover 10 in the present embodiment can be applied to various sensors, such as spectroscopic sensors using ultraviolet or visible light, other optical sensors, sensors using focused beam reflectometry (FBRM) to measure crystal particle size, and electromagnetic sensors to measure dielectric constant or electrical conductivity, and can be combined with these sensors to constitute a sensor device.

As shown in FIG. 3, sensor 12 comprises a rod-shaped sensor probe 12a with a circular cross-section, at the tip portion of which a measurement portion 12b is disposed. Sensor probe 12a passes through an opening formed in the center of flange portion 16 and extends axially into the interior of cover body 14. Sensor probe 12a extends along the center axis of cover body 14, and measurement portion 12b is positioned near the tip portion of cover body 14. Note that in the present embodiment the outer diameter of sensor probe 12a is approximately 20 mm, and is surrounded by cover body 14, which has an outer diameter of approximately 60 mm. It is thus preferable to provide a clearance of approximately 10 mm to approximately 30 mm between sensor probe 12a and the inner wall surface of cover body 14.

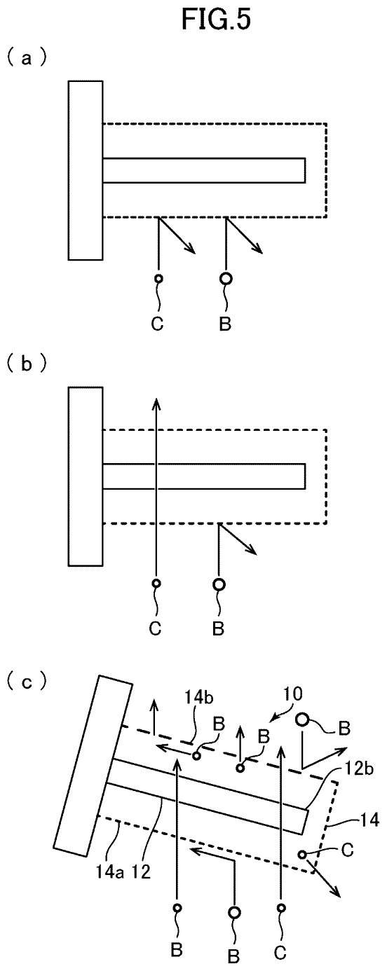

Next, referring again to FIGS. 4 and 5, we explain in detail the constitution of the cover body 14 provided on sensor cover 10 in an embodiment of the present invention.

FIG. 4 shows an enlarged example of micropores formed on the surface of cover body 14. FIG. 5 schematically depicts the operation of the sensor cover.

As described above, numerous micropores are formed over the entire surface of cover body 14 of a sensor device 1 according to an embodiment of the present invention, and the surrounding liquid as well as at least some crystals can flow into the interior of cover body 14. Here, in sensor device 1 of the present embodiment, the size of each micropore formed on the top side of cover body 14 should be set to be equal to or larger than the size of each micropore formed on the bottom side thereof. I.e., a bottom permeable portion 14a which allows liquid to pass through is formed on the bottom surface of cover body 14, and a top permeable portion 14b which allows liquid to pass through is formed on the top surface thereof, whereby the micropores formed in top permeable portion 14b are formed to be the same size or larger than the micropores formed in bottom permeable portion 14a.

As shown in FIG. 2, bottom permeable portion 14a is formed over the entire surface of a bottom semicircular portion corresponding to the bottom semicircle of cover body 14, which has a circular cross-section, and top permeable portion 14b is formed over the entire surface of a top semicircular portion corresponding to the top semicircle on cover body 14. There are also numerous micropores identical to those in bottom permeable portion 14a formed over the entire surface of cover body 14 tip surface 14c.

In the present Specification, the cover body 14 “bottom surface” means the side illuminated when light is projected vertically downward from sensor cover 10 installed in a usage state, and “top surface” means the side illuminated when light is projected in the vertically upward direction therefrom. In the present embodiment numerous micropores are formed over the entire “bottom surface” and “top surface” of cover body 14, but micropores do not necessarily have to be formed over the entire surface; it is sufficient that they be formed in a portion thereof. Furthermore, in the present embodiment micropores are also formed on the tip surface 14c of cover body 14, but it is also acceptable not to form micropores on tip surface 14c.

Next, as shown in FIG. 4, approximately circular micropores are arrayed in a staggered pattern on bottom permeable portion 14a and top permeable portion 14b. I.e., micropores are arrayed so that lines connecting the centers of three adjacent micropores would form an equilateral triangle. In the present embodiment, bottom permeable portion 14a and top permeable portion 14b are constituted to form numerous micropores by etching a thin sheet of stainless steel. As an example, in the present embodiment bottom permeable portion 14a is formed by arraying circular holes with a diameter D=approximately 750 μm at a pitch P=approximately 1070 μm (the length of the sides of an equilateral triangle connecting the centers of the circles). In the present embodiment, top permeable portion 14b is constituted by arraying circular holes with a diameter D=approximately 750 μm at a pitch P=approximately 1070 μm.

Here, bottom permeable portion 14a and top permeable portion 14b, formed by arraying circular holes with a diameter D=approximately 750 μm at a pitch P=approximately 1070 μm results in an opening area of approximately 44.4%, roughly corresponding to a #20 mesh (#20 mesh: a mesh in which 20 strands per inch are longitudinally and transversely disposed). In the present Specification, “micropore size” in the bottom permeable portion 14a and top permeable portion 14b refers to the circular hole diameter.

Note that if circular holes with a diameter D=approximately 540 μm are arrayed at a pitch P=approximately 770 μm, the open area ratio is about 44.5%, roughly corresponding to a #30 mesh. In addition, circular holes of diameter D=approximately 350 μm arrayed at a pitch P=approximately 630 μm would result in an aperture ratio of approximately 28%, roughly corresponding to a #40 mesh. Circular holes of diameter D=approximately 250 μm arrayed at a pitch P=approximately 360 μm would result in an aperture ratio of approximately 43.6%, roughly corresponding to a #60 mesh. Circular holes of diameter D=approximately 180 μm arrayed at a pitch P=approximately 320 μm would result in an aperture ratio of approximately 28.7%, roughly corresponding to a #80 mesh. Circular holes of diameter D=approximately 150 μm arrayed at a pitch P=approximately 250 μm would result in an aperture ratio of approximately 32.5%, roughly corresponding to a #100 mesh, and circular holes of diameter D=approximately 100 μm arrayed at a pitch P=approximately 210 μm would result in an aperture ratio of approximately 20.5%, roughly corresponding to a #150 mesh.

In the present embodiment the bottom permeable portion 14a and top permeable portion 14b are constituted by forming numerous micropores in a thin plate, but a bottom permeable portion 14a or top permeable portion 14b can also be constituted from a mesh-shaped object formed by combining fine strands of wire, such as by weaving, knitting, or the like. In such cases, “micropores” are formed as the spaces between strands constituting the mesh object, and “micropore size” indicates the distance between adjacent strands. The diameter of micropores formed in bottom permeable portion 14a is preferably set to between approximately 180 μm and approximately 750 μm. More preferably, the diameter of micropores in bottom permeable portion 14a is set to between approximately 250 μm and approximately 750 μm. Even more preferably, the diameter of micropores in bottom permeable portion 14a is set to between approximately 350 μm and approximately 750 μm. Even more preferably, the diameter of micropores in bottom permeable portion 14a is set to between approximately 540 μm and approximately 750 μm. I.e., it is preferable to set the diameter of micropores in bottom permeable portion 14a to a size that suppresses the penetration of bubbles into culture liquid L and also allows at least some of the crystals of the culture liquid L to pass through. The bottom permeable portion 14a and top permeable portion 14b, in which numerous micropores are formed in a thin plate, reduce adherence and deposition of crystals, and improve maintainability of the sensor cover.

To facilitate the discharge of bubbles which have entered into the cover body, it is preferable to form the micropores disposed in top permeable portion 14b in a size larger than the micropores disposed in bottom permeable portion 14a. However, the diameter of micropores in top permeable portion 14b should be set to a size fully capable of suppressing the inflow of bubbles through top permeable portion 14b caused by the downward flow of liquid. The diameter of the micropores formed in top permeable portion 14b is preferably about 1 to 5 times the diameter of micropores formed in bottom permeable portion 14a. This allows for an appropriate balance between the suppression of bubbles penetrating into cover body 14 and the discharge of bubbles which do penetrate into cover body 14.

Next, referring to FIG. 5, we explain the operation of a sensor cover.

First, it has been known for some time that when measuring the properties of a liquid into which bubbles are mixed, the effect of the bubbles can be reduced by covering the sensor with a mesh-shaped strainer. However, as shown in FIG. 5(a), when crystals are mixed into the culture liquid L, setting the size of the micropores in cover body 14 (bottom permeable portion 14a, top permeable portion 14b) to be too fine in order to stop the penetration of bubbles will prevent the penetration of both bubbles B and crystals C into the strainer interior. In such cases, components of the crystals cannot be measured by the sensor and accurate measurements cannot be made.

On the other hand, as shown in FIG. 5(b), setting the size of micropores in the cover body 14 covering the sensor to an appropriate size suppresses the penetration of bubbles B while at the same time allowing at least some of the crystals C to penetrate into the interior of cover body 14 so that components of the crystals can be measured by the sensor.

More preferably, as shown in FIG. 5(c), small diameter micropores are formed on bottom permeable portion 14a of cover body 14, and micropores larger than those in bottom permeable portion 14a are formed on top permeable portion 14b. By forming the cover body 14 in this way, penetration into cover body 14 by large bubbles floating up from below can be stopped. At least some of the crystals in culture liquid L enter into cover body 14 through bottom permeable portion 14a to be detected by the sensor. In addition, as shown in FIG. 5(c), by disposing cover body 14 so that it projects diagonally downward from the side wall surface 2a of fermentation vessel 2, bubbles prevented from penetrating by cover body 14 can easily move diagonally upward along the bottom surface of cover body 14, and many of the bubbles B can move upward while easily bypassing sensor cover 10.

On the other hand, small bubbles approaching from below pass through bottom permeable portion 14a and penetrate into cover body 14. However, by forming large-diameter micropores in top permeable area 14b, small bubbles passing through bottom permeable portion 14a and penetrating will float up inside cover body 14 and can be easily discharged to the outside through top permeable portion 14b. Penetrating bubbles can therefore be prevented from accumulating for long periods inside cover body 14 and growing there into large bubbles.

In addition, while not all bubbles approaching sensor cover 10 approach from the bottom side, a buoyancy force from the liquid is always acting on the bubbles, therefore the fraction of bubbles approaching sensor cover 10 from below is high, and their penetration is greatly suppressed by bottom permeable portion 14a. Moreover, top permeable portion 14b more effectively stops the penetration of some bubbles approaching sensor cover 10 from above or from the side, etc. as the result of liquid flow caused by stirring in the fermentation vessel than if the top portion of the cover were completely open. Furthermore, forming larger micropores in top permeable portion 14b allows crystals C to more easily penetrate into the interior of sensor cover 10 so that the crystal component can be more accurately detected.

By disposing cover body 14 so that it projects diagonally downward, bubbles that have penetrated into cover body 14 will move upward toward the base portion of cover body 14. On the other hand, measurement portion 12b on sensor probe 12a is disposed close to the tip of cover body 14, therefore bubbles inside cover body 14 are moved away from sensor 12 measurement portion 12b. This enables the effect on measurement of bubbles entering into cover body 14 to be even further reduced.

Using sensor cover 10 of the present embodiment, these actions effectively reduce the influence of bubbles on measurement.

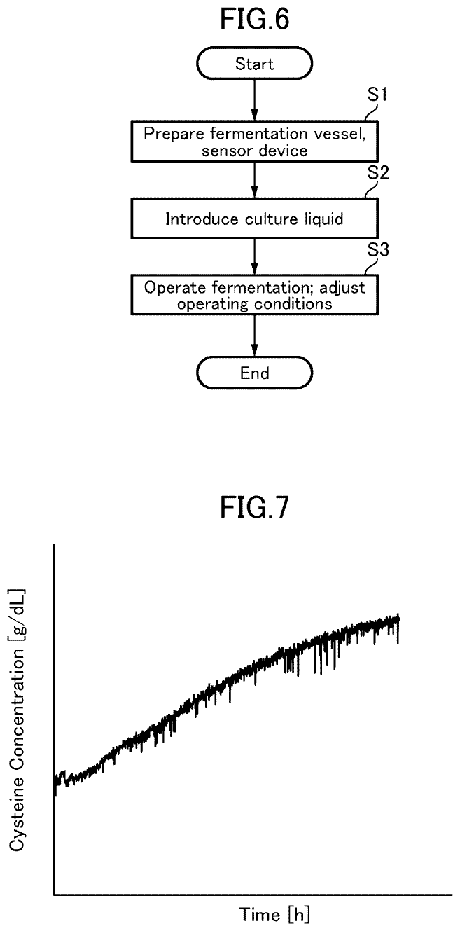

Next, referring to FIGS. 6 through 10, we explain a method for manufacturing fermentation products using a sensor device 1 according to an embodiment of the present invention.

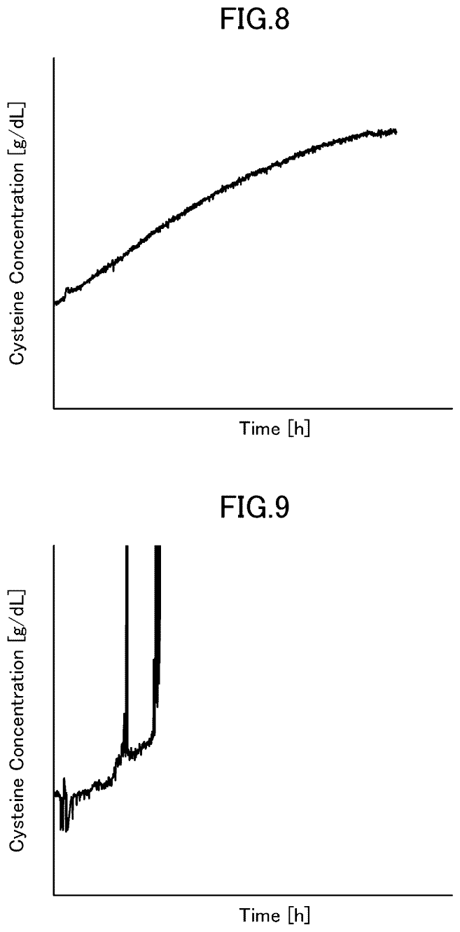

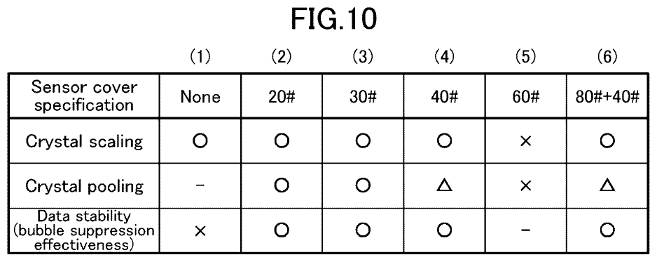

FIG. 6 is a flowchart showing a procedure for manufacturing fermentation products according to an embodiment of the invention. FIG. 7 shows an example of a measurement made without a sensor cover 10. FIG. 8 shows an example of a measurement with a sensor device 1 using an appropriate sensor cover 10. FIG. 9 shows an example of a measurement using a sensor cover 10 in which the micropores are too fine. FIG. 10 also shows the post-use condition of various sensor covers 10.

First, in step S1 of FIG. 6, a fermentation vessel 2 and a sensor device 1 for measuring properties of the liquid in this fermentation vessel 2 are prepared. In this embodiment, a sparger 8 is provided in fermentation vessel 2, and an air supply device 8a is connected to sparger 8. A cover with micropores of suitable size according to the average particle diameter of crystals produced in fermentation vessel 2 is selected for the sensor cover 10 used on the sensor 1 to be applied. Multiple sensor devices 1 can also be installed on a single fermentation vessel 2 in accordance with the required measurement item.

Next, in step S2, liquid to be fermented in fermentation vessel 2 is introduced into fermentation vessel 2. In the example shown here, cysteine is produced as the fermentation product by fermenting the culture liquid L introduced into fermentation vessel 2; at least a portion of this cysteine is oxidized in the culture liquid to produce cystine (Cys2). As an example, culture liquid L contains a carbon source, a nitrogen source, a sulfur source, and inorganic ions.

Sugars such as glucose, fructose, sucrose, molasses, and starch hydrolysates, and organic acids such as fumaric acid, citric acid, succinic acid, and the like can be used as carbon sources.

Inorganic ammonium salts such as ammonium sulfate, ammonium chloride, and ammonium phosphate, organic nitrogen such as soybean hydrolysate, ammonia gas, ammonia water, and the like can be used as nitrogen sources.

Sulfur sources can include inorganic sulfur compounds such as sulfates, sulfites, sulfides, hypo sulfites, thiosulfates, and the like.

It is desirable to include appropriate amounts of required substances such as vitamin B1 or yeast extract as an organic micronutrient source. In addition to these, small amounts of potassium phosphate, magnesium sulfate, iron ions, manganese ions, and the like may be added as needed.

In step S3, fermentation vessel 2, into which culture liquid L is introduced, is operated for fermentation, and properties of the liquid in fermentation vessel 2 are measured by sensor device 1. During the fermentation operation, the culture liquid L in the fermentation vessel 2 is agitated by operating an agitator 6, and by operating a feeder 8a; bubbles are mixed into culture liquid L via sparger 8. In the present embodiment the average diameter of bubbles mixed into culture liquid L by sparger 8 is approximately 250 μm. This volume introduced per hour of this air corresponds to about 0.1 to 1.2 times the volume of the medium in fermentation vessel 2 at the start of fermentation. The volume of air fed into fermentation vessel 2 per hour is preferably less than or equal to 1 to 1.2 times the volume of the medium at the start of fermentation in fermentation vessel 2, so that air can be dissolved in the liquid without excessive fragmentation of the bubbles.

In addition, fermentation operating conditions are adjusted based on measurement results from sensor device 1. In the present embodiment, cystine is produced by the fermentation operation; as the amount of cystine dissolved in culture liquid L increases, it precipitates to form cystine crystals. Properties of culture liquid L are measured from time to time during the fermentation operation by sensor device 1. In the present embodiment, the properties of culture liquid L measured by sensor device 1 include cystine concentration [g/L], sugar concentration [g/L] (R.S.), ammonia nitrogen concentration (AN), S₂0₃ concentration [g/L], turbidity (OD) of culture liquid L, and the like.

In addition, fermentation operating conditions are adjusted based on the properties of culture liquid L measured by sensor device 1. Conditions of the fermentation operation to be adjusted include the culture liquid L temperature, the culture liquid L pH, the amount of aeration fed through sparger 8, the amount of sugar solution added, the amount of each component such as phosphorus added, and the like. Adjustments to fermentation operating conditions based on these measured properties of culture liquid L are performed as needed during the fermentation operation.

We now explain the measurement of cystine concentration by sensor device 1.

As noted above, sensor device 1 comprises a sensor 12 (FIG. 3). In the present embodiment, sensor 12 is an NIR sensor; light received by sensor probe 12a is guided to measurement instrument main unit 4 (FIG. 1) over optical fiber. An NIR spectrum of the guided light is acquired by measurement instrument main unit 4. The concentration of cystine and the like in culture liquid L can be estimated by applying a calibration model prepared in advance to the NIR spectrum obtained for the light from the sensor probe 12a. FIGS. 7 through 9 show graphs of changes with time in cystine concentration estimated in this way.

FIG. 7 shows the cystine concentration in the culture liquid L in fermentation vessel 2 when measured (estimated) using sensor 12 without a sensor cover 10. As shown in FIG. 7, although the concentration of cystine produced in fermentation vessel 2 tends to increases over time, the estimated values fluctuate greatly with each measurement when measuring without a sensor cover 10. It is believed that bubbles mixed into the culture liquid L affect the light received by sensor probe 12a, scattering the estimated values.

FIG. 8 shows an example of the estimated value when the concentration of cystine in culture liquid L as measured in the FIG. 7 example is measured using a sensor device 1 fitted with a sensor cover 10. In the example shown in FIG. 8, a sensor cover 10 on which micropores respectively equivalent to a #20 mesh are formed on bottom permeable portion 14a and top permeable portion 14b. As shown in FIG. 8, when a sensor cover 10 with #20 mesh equivalent micropores is used, the estimated cystine concentration trend is the same as when measured without using a sensor cover 10.

Also, when compared to the FIG. 7 example, the fluctuation in estimated cystine concentration is smaller in the FIG. 8 example, indicating that the cystine concentration is being measured stably. This is thought to be because sensor cover 10 is disposed to surround sensor 12, suppressing the effect of bubbles mixed into culture liquid L and stabilizing the measured value. Also, cystine has low solubility in culture liquid L, and much of the cystine produced in fermentation vessel 2 precipitates and crystallizes. The average particle size of this crystallized cystine is 5 μm or greater, but it is clear that crystalized cystine is being detected even with sensor cover 10 attached to sensor 12. I.e., it is clear that the bottom permeable portion 14a and top permeable portion 14b of sensor cover 10 are passing the culture liquid L and at least some of the crystals in the culture liquid L.

FIG. 9 shows an example in which the cystine concentration in culture liquid L measured in the FIG. 8 example is measured using a sensor cover 10 with smaller micropores than those in FIG. 8. The FIG. 9 example shows the use of a sensor cover 10 with #60 mesh equivalent micropores respectively in the bottom permeable portion 14a and top permeable portion 14b. In the FIG. 9 example, the estimated cystine concentration is stable immediately following the start of fermentation operation, as in the example shown in FIG. 8, but fluctuations in the estimated value increase with the passage of time. As more time passes, anomalies appear in the estimates, such that eventually the cystine concentration can no longer be estimated. This is because during the fermentation operation, scaling of cystine crystals occurs on the outside of sensor cover 10, and crystals accumulate on the inside of sensor cover 10.

I.e., this is believed to be due to the fact that for a fermentation operation in which cystine is produced in a culture liquid L in fermentation vessel 2, the micropores are too fine in a sensor cover with micropores equivalent to a #60 mesh, so that crystals precipitated from the liquid phase cannot be discharged from the cover, resulting in crystal preciptation and deposition. In contrast, in the FIG. 8 example there is essentially no crystal deposition on sensor cover 10 even after a long fermentation operation, indicating that for a fermentation operation to produce cystine, a sensor cover 10 with #20 mesh equivalent micropores is appropriate.

In the examples shown in FIGS. 8 and 9, micropores of the same size were disposed in bottom permeable portion 14a and top permeable portion 14b of sensor cover 10, but the size of micropores in top permeable portion 14b may also be formed to be larger than the micropores in bottom permeable portion 14a. In this case, bubbles penetrating into sensor cover 10 can more easily pass through top permeable portion 14b and be discharged, therefore we may expect the adverse effect on readings caused by penetrating air bubbles to be further reduced. For example, the micropores in top permeable portion 14b of the sensor cover 10 used for measurement as shown in FIG. 8 may be changed to a #10 to #15 mesh equivalent, which is larger than the micropores in bottom permeable portion 14a.

Next, referring to FIG. 10, we explain the deposition of crystals in the case where measurement is conducted using various sensor covers 10.

FIG. 10 is a table summarizing the results of experiments conducted on the state of crystal deposition and the stability of measurement data obtained when various sensor covers 10 were used.

As shown in FIG. 10, experiments were conducted for a condition (1) in which no sensor cover 10 was used, and for conditions (2) through (5), in which #20 mesh equivalent, #30 mesh equivalent, #40 mesh equivalent, and #60 mesh equivalent were respectively used. Experiments were also conducted in which, as condition (6), the bottom permeable part 14a of sensor cover 10 used an #80 mesh equivalent, and the top permeable portion 14b used a #40 mesh equivalent. In the experiment, the state of crystal deposition on sensor 12 and sensor cover 10 was observed for each condition after a predetermined fermentation operation time.

First, as shown in FIG. 10, in condition (1), when no sensor cover 10 was used, adhesion of crystals (crystal scaling) to sensor 12 was not observed, however there was a large fluctuation in estimated cystine concentration, such that data could not be stably measured. I.e., when no sensor cover 10 is provided, crystallized cystine can be measured by sensor 12, but a large amount of noise is introduced into the cystine concentration estimated values due to the effect of bubbles mixed into culture liquid L, causing large fluctuations (hunting) in the estimated values.

Next, when sensor covers 10 with the #20 mesh equivalent of condition (2) and the #20 mesh equivalent of condition (3) were used, no adhesion was found on sensor 12 or the outer surface of sensor cover 10, nor was any crystal accumulation inside sensor cover 10 found. There was also no significant fluctuation in estimated cystine concentrations, and data could be stably measured. I.e., when sensor cover 10 is set to a #20 mesh equivalent or a #30 mesh equivalent, the micropore size is sufficiently larger than the average particle size of cystine crystals, and cystine crystals can sufficiently penetrate into sensor cover 10. Highly reliable estimates of cystine concentration by detection of crystals penetrating into sensor cover 10 using sensor 12 can thus be obtained, and crystals that do penetrate into sensor cover 10 can be easily discharged out of sensor cover 10. On the other hand, most of the bubbles in culture liquid L are prevented from penetrating into sensor cover 10, and the adverse effects of bubbles on sensor 12 are suppressed.

Next, when a sensor cover 10 with a #40 mesh equivalent was used in condition (4), no adhesion of crystals to the outer surface of sensor cover 10 was found, but a small accumulation of crystals was found on the inside of sensor cover 10. Estimated cystine concentrations did not fluctuate significantly during the experimental period, and data could be stably measured. Under these conditions, however, there is a risk that when a fermentation operation is conducted for an extended period, crystal accumulation inside sensor cover 10 may increase, such that proper readings cannot be acquired. When the micropores disposed in sensor cover 10 are small, the penetration of bubbles into sensor cover 10 can in this way be more strongly suppressed, which is advantageous for eliminating the adverse effects of bubbles. On the other hand when micropores are small, cystine crystals which have entered sensor cover 10 and crystals formed inside the cover are difficult to discharge to the outside, and are more prone to accumulate inside sensor cover 10.

Furthermore, when a condition (5) #60 mesh equivalent sensor cover 10 was used, adhesion of crystals to the outer surface of sensor 12 and sensor cover 10, and a large accumulation of crystals inside sensor cover 10, were found. Also, it became impossible to obtain proper data during the experimental period due to the effects of crystals accumulated in sensor cover 10. Effectiveness in suppressing the penetration of bubbles is thus strengthened when a sensor cover 10 with even finer micropores than condition (4) is used, but the amount of cystine crystals accumulating inside sensor cover 10 increases dramatically. When a large quantity of cystine crystals accumulates in sensor cover 10 in this way, cystine concentration inside sensor cover 10 rises dramatically, making it difficult to accurately measure the cystine concentration in the culture liquid L inside fermentation vessel 2.

Finally, no adhesion of crystals to the outside surface of sensor 12 or sensor cover 10 was found when an #80 mesh equivalent was used for bottom permeable portion 14a and a #40 mesh equivalent was used for top permeable portion 14b of sensor cover 10 under condition (6), but a small accumulation of crystals was found inside sensor cover 10. The estimated cystine concentrations did not fluctuate significantly during the experimental period, and data could be stably measured. Under these conditions, however, there is a risk that when the fermentation operation is conducted for an extended period, crystal accumulation inside sensor cover 10 may increase, such that proper readings cannot be acquired. Also, a better result was obtained in condition (6) than in condition (5), even though micropores in bottom permeable portion 14a were set at an #80 mesh equivalent, which is finer than in condition (5). I.e., it is believed that because the micropores in top permeable portion 14b are larger in condition (6) than in condition (5), crystal accumulation inside sensor cover 10 is less likely to occur. Therefore a sensor cover 10 with which data can be stably measured over a long time period, while also further reducing the adverse effects of bubbles can, by fine tuning the size of the micropores in the sensor cover 10 under condition (6), be constructed using a bottom permeable portion 14a with fine micropores.

From the results shown in FIG. 10 it was found that a sensor cover 10 having micropores equivalent to #20 mesh and #30 mesh was appropriate for fermentation operations to produce cystine in a culture liquid L in fermentation vessel 2. FIG. 10 shows the results when cystine is manufactured as a fermentation product in a fermentation vessel 2, but when other crystal producing fermentation products are produced, a sensor cover 10 furnished with micropores appropriately sized to the average particle size of the crystals produced and the average diameter of bubbles mixed into L should be designed.

According to the method of producing fermentation products of the present embodiment of the invention, a sensor device 1 for measuring properties of culture liquid L, which is the liquid in fermentation vessel 2, has a cover body 14 disposed to surround sensor 12. A bottom permeable portion 14a and top permeable portion 14b which pass culture liquid L and at least part of the crystals in culture liquid L are disposed on cover body 14, therefore the culture liquid L to be measured can continuously flow into and out of cover body 14. As a result, the culture liquid L in cover body 14 is constantly replaced, and the sensor 12 disposed inside cover body 14 can measure properties of the culture liquid L containing crystals continuously and in real time.

In addition, because bottom permeable portion 14a and top permeable portion 14b of cover body 14 pass at least a portion of crystals in culture liquid L, those crystals which form in culture liquid L are less likely to accumulate in cover body 14, and the fermentation operation can be continued for a long without the need for maintenance such as cleaning the cover body 14. The micropores formed in top permeable portion 14b are larger than those formed in bottom permeable portion 14a, therefore bubbles which float up from the bottom of cover 14 are less prone to pass through the micropores in bottom permeable part 14a, and penetration of bubbles into cover body 14 can be effectively suppressed. On the other hand the micropores in top permeable portion 14b are formed to be the same or larger than those in bottom permeable portion 14a, therefore bubbles which penetrate into cover body 14 can easily float up in cover body 14 to be discharged through top permeable portion 14b. As a result, bubbles penetrating cover body 14 accumulate inside cover body 14, so their adverse effects on sensor 12 readings can be suppressed.

According to the fermentation product manufacturing method of the present embodiment, the average diameter of bubbles mixed into culture liquid L in fermentation vessel 2 is 250 μm, therefore cover body 14 can suppress the penetration of bubbles while also allowing the passage of crystals produced in the liquid with an average particle size of 5 μm or greater. Stable measurements can thus be obtained by sensor 12.

In addition, according to the fermentation product manufacturing method of the present embodiment, the hole diameter at the outlet of the ventilation tube for feeding gas into fermentation vessel 2 is 6 mm, therefore the average diameter of bubbles mixed into culture liquid L in fermentation vessel 2 increases. Therefore cover body 14 suppresses the penetration of bubbles while allowing crystals produced in culture liquid L with an average particle size of 5 μm or greater to pass through. Stable measurements can thus be obtained by sensor 12.

According to the fermentation product manufacturing method of the present embodiment, the volume per hour of air, which is the gas being fed into fermentation vessel 2 through sparger 8, a ventilation tube, is between 0.1 to 1.2 times the culture medium volume at the start of fermentation, therefore the air can be dissolved into culture liquid L without excessive fragmentation of the air bubbles fed into fermentation vessel 2. As a result, bubbles can be easily suppressed from penetrating into cover body 14.

According to the fermentation product manufacturing method of the present embodiment, cover body 14 is furthermore formed in an approximately cylindrical shape (FIG. 2), therefore bubbles floating up from the bottom side of cover body 14 can easily flow upward along the lower surface of cover body 14, further suppressing the penetration of bubbles into cover body 14. Because cover body 14 is formed in an approximately cylindrical shape, bubbles penetrating into cover body 14 and floating up are collected at the highest point therein, and are less prone to contact sensor 12. This minimizes adverse effects on measurement even when bubbles do penetrate the cover body 14.

According to the fermentation product manufacturing method of the present embodiment, a bottom permeable portion 14a and top permeable portion 14a are respectively located over the entire surfaces of the lower and upper semicircular portions, therefore the portion of cover body 14 through which the culture liquid L and crystals can permeate can be made extremely large. As a result, crystals which have penetrated into cover body 14 can be easily discharged to the outside, and accumulation of crystals in the cover body 14 can be suppressed.

Furthermore, according to the method for manufacturing fermentation products of the present embodiment, micropores are formed in bottom permeable portion 14a and top permeable portion 14b by the holes provided in a thin metal plate (FIG. 4), therefore compared to the case in which a bottom permeable portion 14a or top permeable portion 14b is formed by a mesh object made by weaving strands, crystals are less prone to adhere and deposit on bottom permeable portion 14a and top permeable portion 14b, and maintenance properties of the sensor cover 10 can be improved. Forming micropores in bottom permeable portion 14a and top permeable portion 14b by providing holes in a thin metal plate enables constitution of a cover body 14 less susceptible to breakage than a mesh-shaped object, with high durability.

In the method for manufacturing fermentation products of the present embodiment, when the diameter of micropores in top permeable portion 14b is larger than the diameter of micropores in bottom permeable portion 14a, the discharge of crystals and bubbles within the cover body can be promoted, while the entry of bubbles into cover body 14 is effectively suppressed, therefore the influence of accumulated crystals and bubbles on sensor 12 can also be effectively suppressed.

Furthermore, according to the method for manufacturing fermentation products of the present embodiment, the diameter of micropores formed on bottom permeable portion 14a is from 180 μm to 750 μm, therefore the penetration of large bubbles which are prone to adversely affect sensor 12 readings can be effectively suppressed, while the inflow of liquid and crystals into cover body 14 is allowed.

According to the fermentation product manufacturing method of the present invention, cover body 14 projects diagonally downward (FIG. 3) from the side wall surface 2a of a fermentation vessel 2 containing a culture liquid L, therefore bubbles floating up from below and reaching cover body 14 can easily flow upward along the underside of cover body 14, so that penetration thereof into cover body 14 can be effectively suppressed. Bubbles penetrating into cover body 14 collect at the base portion of cover body 14 and are therefore distant from measurement portion 12b on sensor 12, which is positioned near the tip portion of cover body 14 located above the cover body 14, thus reducing their adverse effect on measurement.

We have explained preferred embodiments of the present invention above, however various changes may be applied to the above-described embodiments. In particular, in the above-described embodiment of the invention, cystine was produced as the fermentation product in a culture liquid L into which bubbles were mixed, but the invention may be applied to the manufacture of any other desired fermentation product in which crystals are produced in a liquid into which bubbles are mixed. For example, the invention may be applied to fermentation vessels where amino acids, nucleic acids, or peptides, which have relatively low solubility and from which crystals can easily precipitate, are produced in a culture liquid L in which fermentation products, fermentation carbon sources, nitrogen sources, phosphorus sources, oxygen sources, and/or sulfur sources are dissolved.

Here fermentation products include, for example, amino acids; fermentation carbon sources include, for example, sugars, monosaccharides, disaccharides, and polysaccharides; nitrogen sources include, for example, ammonia and ammonium sulfate; phosphorus sources include, for example, phosphate ions; and sulfur sources include, for example, thiosulfate ions. Examples of amino acids, nucleic acids, and peptides whose crystals are relatively easy to precipitate during fermentation include glutamic acid (Glu), tryptophan (Trp), phenylalanine (Phe), threonine (Thr), tyrosine (Tyr), cysteine (Cys), cystine (Cys2), glutamine (Gln), aspartic acid (Asp), leucine (Leu), isoleucine (Ile), valine (Val), inosine, guanosine, adenine, and others.

In the embodiments described above, measurement was made of the concentration of components precipitated in the liquid, but it is also possible to measure the properties of the liquid itself, such as its dielectric constant or electrical conductivity, or to measure bacteria in the liquid.

Furthermore, in the above-described embodiment of the invention, a single sensor device was installed in fermentation vessel 2 to measure various properties of the liquid, but it is also possible to install multiple sensor devices 1 in fermentation vessel 2. In such cases, different covers for sensors may be applied to each sensor device according to the characteristic measured by each sensor device. For example, one sensor device can measure properties of precipitated crystals, while another sensor measures properties of components dissolved in the liquid phase.

### EXPLANATION OF REFERENCE NUMERALS

1 sensor device
2 fermentation vessel
2a side wall surface
4 instrument main unit
6 agitator
6a blades
8 sparger (vent pipe)
8a feed device
10 sensor cover
12 sensor
12a sensor probe
12b measurement portion
14 cover body
14a bottom permeable portion
14b top permeable portion
14c tip surface
16 flange portion
18a affixing bolt
18b packing

1. A method for manufacturing an amino acid by operation of a fermentation vessel bubbles are mixed and crystals with an average particle size of 5 μm or greater are formed in a liquid, comprising:
preparing a fermentation vessel and a sensor device for measuring properties of liquid inside the fermentation vessel;
introducing liquid to be fermented into the fermentation vessel; and
operating the fermentation vessel for fermentation, wherein properties of liquid in the fermentation vessel are measured by the sensor device, and conditions of fermentation operation are adjusted based on results of the measurement;
wherein the sensor device has a sensor for measuring liquid properties and a cover body for the sensor disposed to surround the sensor;
wherein the cover body has a bottom side permeable portion for passing at least a portion of the liquid and crystals in the liquid disposed on at least a portion of the bottom surface of the cover body and a top side permeable portion for passing at least a portion of the liquid and crystals in the liquid disposed on at least a portion of the top surface of the cover body; and
wherein the bottom side permeable portion and the top side permeable portion have numerous micropores for passing liquid respectively, and the micropores disposed on the top side permeable portion are sized to be the same as or larger than the micropores disposed on the bottom side permeable portion.

2. The method for manufacturing the amino acid of claim 1, wherein the average diameter of bubbles mixed into the liquid in the fermentation vessel is 50 μm or greater.

3. The method for manufacturing the amino acid of claim 1, wherein the fermentation vessel comprises a ventilation tube for feeding gas into the fermentation vessel, and the hole diameter at an outlet of the ventilation tube is 1 μm or larger.

4. The method for manufacturing the amino acid of claim 1, wherein the fermentation vessel comprises a ventilation tube for feeding gas into the fermentation vessel, and the volume of gas fed into the fermentation vessel per hour is less than or equal to twice the culture medium volume at a start of fermentation in the fermentation vessel.

5. The method for manufacturing the amino acid of claim 1, wherein the cover body is formed in a cylindrical shape, and the sensor extends into interior thereof in an axial direction.

6. The method for manufacturing the amino acid of claim 5, wherein the bottom permeable portion is disposed over the entire surface of a lower semicircular portion of the approximately cylindrical cover body, and the top permeable portion is disposed over the entire surface of an upper semicircular portion of the cover body.

7. The method for manufacturing the amino acid of claim 1, wherein the cover body is formed from a thin metal sheet, and the micropores disposed in the bottom permeable portion and top permeable portion are approximately circular holes formed in the thin metal sheet.

8. The method for manufacturing the amino acid of claim 1, wherein the diameter of micropores disposed in the top permeable portion is 1 to 5 times larger than the diameter of micropores disposed in the bottom permeable portion.

9. The method for manufacturing the amino acid of claim 1, wherein the diameter of micropores disposed in the bottom permeable portion is between 540 μm and 750 μm.

10. The method for manufacturing the amino acid of claim 1, wherein the amino acid produced by the fermentation operation of the fermentation vessel is cysteine, and oxidation of at least part of the cysteine results in the accumulation of cystine in the fermentation vessel.

11. The method for manufacturing the amino acid of claim 1, wherein the cover body is disposed to project diagonally downward from the sidewall surface of the fermentation vessel containing liquid, and a measurement portion of the sensor is positioned near a tip portion of the cover body.

12. The method for manufacturing the amino acid of claim 1, wherein said amino acid is at least one member selected from the group consisting of glutamic acid (Glu), tryptophan (Trp), phenylalanine (Phe), threonine (Thr), tyrosine (Tyr), cysteine (Cys), cystine (Cys2), glutamine (Gln), aspartic acid (Asp), leucine (Leu), isoleucine (Ile), valine (Val), inosine, guanosine, and adenine.
